(19) United States
(12) Patent Application Publication
(10) Pub. No.: US 2023/0181922 A1
(43) Pub. Date: Jun. 15, 2023

(54) NASAL DISEASE TREATMENT AID DEVICE

(71) Applicant: AJOU UNIVERSITY INDUSTRY-ACADEMIC COOPERATION FOUNDATION, Suwon-si (KR)

(72) Inventor: In Su PARK, Seongnam-si (KR)

(73) Assignee: AJOU UNIVERSITY INDUSTRY-ACADEMIC COOPERATION FOUNDATION, Suwon-si (KR)

(21) Appl. No.: 17/925,117

(22) PCT Filed: May 4, 2021
(86) PCT No.: PCT/KR2021/005599

§ 371 Date: Nov. 14, 2022

(30) Foreign Application Priority Data
May 13, 2020 (KR) 10-2020-0057133

(51) Int. Cl. A61N 5/06; A61N 1/04; A61N 1/36

(52) U.S. Cl. CPC A61N 5/0603; A61N 1/0492; A61N 1/36014; A61N 2005/0607; A61N 2005/0659; A61N 2005/0651; A61N 7/00

(57) ABSTRACT

The present invention provides a nasal disease treatment aid device, comprising: a nose cover which forms a dome so as to cover the nose of a user and covers the nose while being positioned on the nose of the user; a fixing member which is connected to the left and right sides of the nose cover and fixed to the head of the user so that the nose cover is fixed to the nose of the user; insertion members having, interposed therebetween, a support body provided in the inner space of the nose cover and extending outward from the left and right sides of the support body and are formed in tubular shapes each having a hollow inside that communicates back and forth so as to allow the user to breathe through the nostrils; LED light sources which are provided on one side of each of the insertion members and irradiate therapeutic light into the inside of the nose; and low-frequency stimulation units which are provided on each of the left and right sides of the nose cover and physically stimulate paranasal sinuses while in contact with the paranasal sinuses that are around the nose. Therefore, it is possible to provide necessary help for the treatment of rhinitis and paranasal sinusitis at the same time, and infrared treatment light of a corresponding wavelength using the LED light sources and heat treatment by radiation heat from the infrared light are carried out in parallel such that the resulting light and heat is propagated deep into a nasal cavity so as to be able to effectively improve blood circulation, thereby requiring a shorter treatment time and minimizing side effects such as skin rashes and so on caused by prolonged exposure to light and heat.

### TECHNICAL FIELD

The present invention relates to a nasal disease treatment aid device, and more particularly, to a nasal disease treatment aid device which is mounted on a patient's nose to irradiate infrared light, which therapeutic light, into the nose and to selectively apply low-frequency (or ultrasound) stimulation to paranasal sinuses positioned at left and right sides of the nose, thereby assisting in treating rhinitis and paranasal sinusitis.

### BACKGROUND ART

In general, the representative causes of allergic rhinitis are house dust mites, pollen, moulds, pet hair and dandruff, and cockroach fragments which are inhaled through the respiratory tract. In addition, allergic rhinitis can also be caused by food, food additives, and drugs.

As general allergic rhinitis methods, there are an allergen avoidance method of finding and avoiding materials that cause allergies, which is the most important and best treatment and prevention method, and a symptomatic treatment method of treating symptoms using a drug such as an antipyretic drug, a vasoconstrictor, an antihistaminic, or and an adrenocortical hormone drug

However, such treatment methods are easy to use and convenient, but there are concerns about side effects due to frequent use and misuse or abuse, and in some cases, when the treatment methods are used for five days or more, it may cause drug rhinitis. Thus, the user's special attention is required.

That being so, as more improved technology, a device that directly injects saline or drugs into an affected area of a nose has been emerged.

When a tubular flexible container containing a therapeutic agent of saline, benzethonium chloride, alcohol, or the like is purchased to be put into a nose and compressed, the therapeutic agent in a particulate state is directly sprayed onto an affected area of the nose to express an effect of cleaning and treating the affected area.

However, even in such a method, an excessive amount of drug may be consumed every time the tubular flexible container is used, and in particular, foreign materials in an affected area of a nose stimulate the nose in the form in which a liquid drug is directly sprayed, which may cause another nasal disease. Thus, relatively many children or elderly people with nasal diseases avoid using tubular flexible container.

In addition, when a drug is used for the affected tissue for a long time, there is a risk of nose being damage due to severe irritation.

As the related art, Korean Patent Publication No. 10-2009-0119422 (published on Nov. 10, 2009) may be referenced for.

### DISCLOSURE

### [Technical Problem]

In general, for sinusitis treatment, rhinitis treatment, paranasal sinus treatment, and treatment to drain pus caused by bacterial infection should be performed together. To the end, the present invention is directed to providing a nasal disease treatment aid device which is mounted on a patient's nose to irradiate infrared light into the nose and to apply low-frequency (or ultrasound) stimulation to paranasal sinuses, thereby assisting in treating rhinitis and paranasal sinusitis at the same time and concurrently performs heat treatment through infrared therapeutic light having a relevant wavelength using a light-emitting diode (LED) light source and heat treatment through infrared radiant heat to transmit light and heat to a deep nasal cavity, thereby effectively improving blood circulation, reducing at treatment time, and minimizing side effects such as skin rash caused by prolonged exposure to light and heat.

### [Technical Solution]

A nasal disease treatment aid device according to the present invention includes a nose cover which forms a dome shape to cover a nose of a user and is positioned on the nose of the user to cover the nose, a fixing member to which a left side and a right side of the nose cover are connected and which is fixed to a head of the user to fix the nose cover to the nose of the user, an insertion member including a pair of infrared insertion portions which are formed to extend outward from a left side and a right side of a support, which is provided in an inner space of the nose cover, with the support interposed therebetween, and are inserted into nostrils, wherein the infrared insertion portion is formed in a hollow tubular shape of which a front side and a right side are open such that the user breathes through nostrils during use of a device, a light-emitting diode (LED) light source which is provided at one side of the insertion member and irradiates therapeutic light into the nose, and a low-frequency stimulation unit which is provided at each of the left and right sides of the nose cover and physically stimulates a paranasal sinus in contact with the paranasal sinus that is around the nose.

The low-frequency stimulation unit according to the present invention may include an adhesive sheet so as to be in close contact with a face of the user at a sinus position.

The insertion member according to the present invention may have a light diffusion surface formed on one side of an outer peripheral surface thereof to allow the therapeutic light to be diffused and irradiated into the nose.

The infrared insertion portion of the insertion member according to the present invention may be formed in a coil shape.

The LED light source according to the present invention may be formed as an LED device which irradiates infrared light having a relevant wavelength.

### [Advantageous Effects]

A nasal disease treatment aid device according to the present invention has the following effects.

The nasal disease treatment aid device is mounted on a patient's nose to irradiate infrared light (or a light-emitting diode (LED) laser) into the nose and to apply low-frequency (or ultrasound) stimulation to paranasal sinuses, thereby treating rhinitis and paranasal sinusitis at the same time.

The nasal disease treatment aid device forms an open dome shape to cover a nose of a user. An infrared insertion portion is formed in a hollow tubular shape of which a front side and a right side are open such that the user breathes through nostrils. Thus, the user can breathe through a nose even during use of the device.

Heat treatment through infrared therapeutic light having a relevant wavelength using an LED light source and heat treatment through infrared radiant heat are concurrently performed to transmit light and heat to a deep nasal cavity, thereby effectively improving blood circulation, reducing a treatment time, and minimizing side effects such as skin rash caused by prolonged exposure to light and heat.

For sinusitis treatment, rhinitis treatment, paranasal sinus treatment and treatment to drain pus caused by bacterial infection should be performed together, it is possible to assist in treating rhinitis and paranasal sinusitis at the same time.

### DESCRIPTION OF DRAWINGS

FIG. 1 shows exemplary views illustrating a nasal disease treatment aid device and an insertion member according to an embodiment of the present invention.

FIG. 2 shows exemplary views illustrating a nasal disease treatment aid device and an insertion member according to another embodiment of the present invention.

FIG. 3 is an exemplary view illustrating an example of use of a nasal disease treatment aid device according to an embodiment of the present invention.

### BEST MODE OF THE INVENTION

The present invention provides a nasal disease treatment aid device including a nose cover which forms a dome shape to cover a nose of a user and is positioned on the nose of the user to cover the nose, a fixing member to which a left side and a right side of the nose cover are connected and which is fixed to a head of the user to fix the nose cover to the nose of the user, an insertion member having hollow tubular shapes which are formed to extend outward from a left side and a right side of a support, which is provided in an inner space of the nose cover, with the support interposed therebetween and has a front side and a right side that are open such that the user breathes through nostrils, a light-emitting diode (LED) light source which is provided at one side of the insertion member and irradiates therapeutic light into the nose, and a low-frequency stimulation unit which is provided at each of the left and right sides of the nose cover and physically stimulates a paranasal sinus in contact with the paranasal sinus around the nose.

### [Modes of the Invention]

Hereinafter, exemplary embodiments of the present invention will be described in detail with reference to the accompanying drawings. Prior to the description, it should be understood that the terms or words used in the present specification and the appended claims should not be construed as limited to general and dictionary meanings, but interpreted based on the meanings and concepts corresponding to technical aspects of the present disclosure on the basis of the principle that the inventor is allowed to define terms appropriately for the best explanation.

Therefore, the embodiments described in the specification and the configurations shown in the drawings are merely most exemplary embodiments of the present invention and do not represent all the technical ideas of the present invention. Thus, it is to be understood that there may be various equivalents and variations in place of them at the time of filing the present application.

The present invention relates to a nasal disease treatment aid device which selectively irradiates infrared light, which is therapeutic light, into a nose and selectively applies low-frequency (or ultrasound) stimulation to paranasal sinuses positioned at a left side and a right side of the nose, thereby assisting in treating rhinitis and paranasal sinusitis. The present invention will be described below with reference to the accompanying drawings.

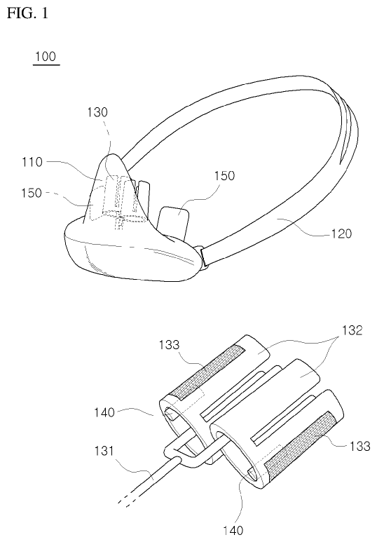

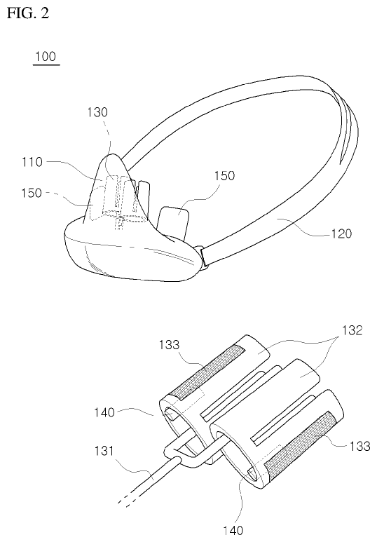

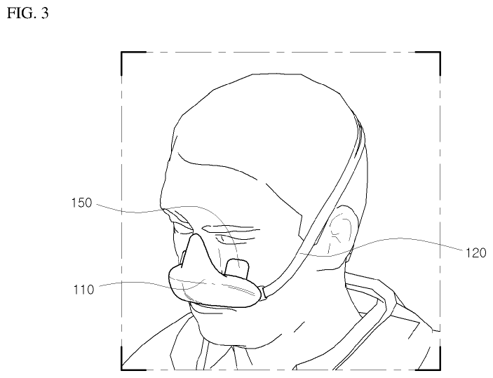

Referring to FIGS. 1 to 3, a nasal disease treatment aid device 100 according to one embodiment of the present invention includes a nose cover 110, a fixing member 120, an insertion member 130, an LED light source 140, and a low-frequency stimulation unit 150. First, the nose cover 110 has a dome shape to cover a nose of the user and is positioned on the nose of the user to cover the nose of the user.

In this case, the nose cover 110 has a dome shape such that the nose of the user may be accommodated in an inner space. Preferably, the nose cover 110 may have an open dome shape in a form of a nose to cover the nose of the user. An elastic band made of a sponge or the like may be provided along a circumference of the nose cover 110, and the circumference of the nose cover 110 may be in close contact with a face of the user to seal the inner space of the nose cover 110 from the outside. A plurality of through-holes communicating with the outside are formed in a lower portion of the nose cover 110 such that the user can breathe.

The insertion member 130 is provided in the inner space of the nose cover 110 so that the nose cover 110 covers the node of the user and the insertion member 130 is inserted into the nose.

In addition, a fixing member 120 is provided for the nose cover 110. The fixing member 120 is connected to left and right sides of the nose cover 110 and is fixed to a head of the user to fix the nose cover 110 to the nose of the user.

Here, any type capable of fixing the nose cover 110 to the head of the user may be applied as the fixing member 120. For example, a headband type fixed behind the head of the user and an earring type hung on and fixed to an ear of the user may be applied.

The insertion member 130 provided in the inner space of the nose cover 110 includes a support 131 and infrared insertion portions 132. The infrared insertion portions 132 are inserted into nostrils of the user are formed at left and right sides of the support 131 with the support 131, which is positioned at a nose post, interposed therebetween.

In this case, it is preferable that the support 131 and the infrared insertion portion 132 of the insertion member 130 are made of a rubber or synthetic resin material that is harmless to a human body and has elasticity, and it is preferable that the infrared insertion portion 132 is formed in a hollow tubular shape of which a front side and a rear side are open such that the user can breathe through the nostrils.

Here, it is preferable that the infrared insertion portion 132 of the insertion member 130 is formed in a tubular shape of which one side is cut in a length direction or is formed in a coil shape such that the infrared insertion portion 132 is easily inserted into or drawn out of the nostril of the user and expands an inner diameter of the nostril.

More specifically, in a case in which the infrared insertion portion 132 of the insertion member 130 is formed in the tubular shape of which one side is cut in the length direction, when the infrared insertion portion 132 is inserted into the nostril, a diameter of the infrared insertion portion 132 is selectively decreased through a cut portion, and thus the infrared insertion portion 132 is easily inserted into the nostril. In addition, in the nostril, the infrared insertion portion 132 expands the inner diameter of the nostril while being restored to the original state thereof by the elasticity of the infrared insertion portion 132.

In a case in which the infrared insertion portion 132 of the insertion member 130 has the coil shape, when the infrared insertion portion 132 is inserted into the nostril, the diameter of the infrared insertion portion 132 is selectively decreased by the self-elasticity, and thus the infrared insertion portion 132 is easily inserted into the nostril. In addition, in the nostril, the infrared insertion portion 132 expands the inner diameter of the nostril while being restored to the original state thereof by the elasticity of the infrared insertion portion 132.

In addition, the LED light source 140 is provided at one side of the insertion member 130. When the insertion member 130 is inserted into the nostril of the user, the LED light source 140 irradiates therapeutic light having a relevant wavelength into the nose.

In this case, the irradiated therapeutic light is infrared light, and when infrared light having a relevant wavelength is irradiated into a nasal cavity, which is the inside of the nose, infrared radiant heat is also irradiated onto an affected area, and thus photothermal effect of the LED light source 140 promotes blood circulation in the nasal cavity to assist in treating rhinitis.

Therefore, the LED light source 140 is an LED light device for irradiating infrared light having a relevant wavelength, and it is preferable that the he LED light source 140 is embedded in the infrared insertion portion 132 of the insertion member 130. It is preferable that a light diffusion surface 133 is formed on one side of the insertion member 130, on which the LED light source 140 is provided, such that therapeutic light is diffused and irradiated into the nose.

The a LED light device that is the LED light source 140, has stronger straightness than a general light source. Therapeutic light irradiated from the LED light source 140 is dispersed and diffused through the light diffusion surface 133, thereby allowing light intensity of the therapeutic light to be uniform and expanding an irradiation range.

The low-frequency stimulation unit 150 is provided at each of the left and right sides of the nose cover 110. The low-frequency stimulation units 150 are in the form of a panel and physically stimulate paranasal sinuses in contact with the paranasal sinuses that are around the nose, thereby assisting in treating paranasal sinusitis.

In this case, it is preferable that an adhesive sheet 151 is provided on a surface of the low-frequency stimulation unit 150 facing the nasal cavity to bring the low-frequency stimulation unit 150 into close contact with a face of the user at a sinus position.

In addition, the low-frequency stimulation unit 150 may be replaced with an ultrasound panel to perform physical stimulation using ultrasound.

Therefore, the nasal disease treatment aid device of the present invention having such a configuration according to the present invention is mounted on a patient's nose to irradiate infrared light into the nose and to apply low-frequency (or ultrasound) stimulation to paranasal sinuses, thereby assisting in treating rhinitis and paranasal sinusitis at the same time. Heat treatment through infrared therapeutic light having a relevant wavelength using an LED light source and heat treatment through infrared radiant heat are concurrently performed to transmit light and heat to a deep nasal cavity, thereby effectively improving blood circulation, reducing a treatment time, and minimizing side effects such as skin rash caused by prolonged exposure to light and heat.

A control unit for controlling the driving of the LED light source 140 and the low-frequency stimulation unit 150 is embedded and provided inside the nose cover 110. The control unit includes a battery and controls power applied to the LED light source 140 and the low-frequency stimulation unit 150, thereby controlling the driving of the LED light source 140 and the low-frequency stimulation unit 150.

In addition, since the control unit is capable of wireless communication, the user may manually operate the setting of the device and the driving and treatment intensity of the LED light source 140 and the low-frequency stimulation unit 150 through a separate remote control.

While the present invention has been described with reference to embodiments shown in the drawings, these should be considered in a descriptive sense only, and it will be understood by those skilled in the art that various alterations and other equivalent embodiments may be made. Therefore the true technical protection scope of the present invention should be determined only by the appended claims.

1. A nasal disease treatment aid device comprising:
a nose cover which forms a dome shape to cover a nose of a user and is positioned on the nose of the user to cover the nose:
a fixing member to which a left side and a right side of the nose cover are connected and which is fixed to a head of the user to fix the nose cover to the nose of the user;
an insertion member including a pair of infrared insertion portions which are formed to extend outward from a left side and a right side of a support, which is provided in an inner space of the nose cover, with the support interposed therebetween;
a light-emitting diode (LED) light source which is provided at one side of the insertion member and irradiates therapeutic light into the nose; and
a low-frequency stimulation unit which is provided at each of the left and right sides of the nose cover and physically stimulates a paranasal sinus in contact with the paranasal sinus that is around the nose.

2. The nasal disease treatment aid device of claim 1, wherein the low-frequency stimulation unit includes an adhesive sheet so as to be in close contact with a face of the user at a sinus position.

3. The nasal disease treatment aid device of claim 1, wherein the insertion member has a light diffusion surface formed on one side of an outer peripheral surface thereof to allow the therapeutic light to be diffused and irradiated into the nose.

4. The nasal disease treatment aid device of claim 3, wherein the infrared insertion portion of the insertion member is formed in a coil shape.

5. The nasal disease treatment aid device of claim 3, wherein the LED light source is formed as an LED device which irradiates infrared light having a relevant wavelength.
